(19) United States
(12) Patent Application Publication
(10) Pub. No.: US 2011/0071333 A1
(43) Pub. Date: Mar. 24, 2011

(54) PROCESS FOR NEUTRALIZATION OF A CATIONIC ZEOLITE

(75) Inventors: Karin Barthelet, Lyon (FR); Patrick Magnoux, Poitiers (FR); Alain Methivier, Lyon (FR); Vania Santos-Moreau, Lyon (FR)

(73) Assignee: IFP Energies Nouvelles, Rueil-Malmaison Cedex (FR)

(21) Appl. No.: 12/994,202

(22) PCT Filed: May 5, 2009
(86) PCT No.: PCT/FR2009/000530

§ 371 Date: Nov. 23, 2010

(30) Foreign Application Priority Data
May 28, 2008 (FR) 0802950

(51) Int. Cl. C10L 3/06; C10G 29/04; C07C 7/13

(52) U.S. Cl. 585/820; 208/297; 208/245; 48/197 FM; 423/701

(57) ABSTRACT

The invention relates to a process for neutralization of a cationic zeolite that is at least partially exchanged with one or more monovalent and/or multivalent cations. The neutralization process comprises at least the stages for dissolution of a basic salt in an anhydrous organic solvent, degassing this solution by bubbling a dry inert gas, suspending the zeolite in this solution under dry inert gas, filtering and washing the solid by an anhydrous organic solvent, and calcination in the presence of oxygen and under a dry gaseous stream. The invention also relates to the use of neutralized zeolites for the separation or the purification of hydrocarbon feedstocks.

### PRIOR ART

Among the processes for separation or purification, there are quite a number that use zeolites in cationic as well as adsorbent form. Their principle resides either in a selectivity of shape or size or in a particular affinity of one of the constituents of the feedstock for the cations. The latter are to be avoided to the extent that the process does not involve any chemical reaction. Any transformation of compounds of the feedstock actually leads to a reduction in yield and can also be at the origin of the formation of coke precursors, thus producing a premature aging of the adsorbent. These undesirable phenomena are all the more frequent as the zeolite has active surface sites that are most often acid sites. Therefore, contrary to the protonated zeolites, the cationic zeolites that do not have a priori Brönsted acid sites should not have strong activity. Nevertheless, in some cases, they have non-negligible activities that are characterized by reactions that involve acid sites.

To be able to obtain zeolites that are very sparingly reactive or even non-reactive, it is necessary either to find a particular preparation method, such as, for example, the activation under reducing atmosphere such as NH₃, which makes it possible to neutralize the protons as soon as they form (H. Siegel, R. Schollner, B. Staudte, J. J. Van Dun, W. J. Mortier, Zeolites, 1987, 7, 372) or to find a means for neutralizing the detected activity. However, in addition to the fact that the zeolites that are obtained by activation under NH₃ are likely to contain NH₃ molecules and therefore to not have their entire porosity accessible, the neutralization is the only conceivable option when it is a matter of working with a zeolite that is provided commercially. However, the neutralization with aqueous basic solutions, conventionally used by one skilled in the art, is not always effective.

To remedy this, a process for neutralization of the zeolite, in particular by using an anhydrous, organic basic solution, is proposed within the framework of this invention. Such an implementation proves effective, surprisingly enough, for limiting, and even cancelling the reactivity of cationic zeolites that are at least partially exchanged with one or more monovalent and/or multivalent cations.

### SUMMARY DESCRIPTION OF THE INVENTION

The invention relates to a process for neutralization of a cationic zeolite that is at least partially exchanged with one or more monovalent and/or multivalent cations. The neutralization process comprises at least the stages for dissolution of a basic salt in an anhydrous organic solvent, degassing this solution by bubbling a dry inert gas, suspending the zeolite in this solution under dry inert gas, filtering and washing the solid by an anhydrous organic solvent, and calcination in the presence of oxygen and under a dry gaseous stream. The invention also relates to the implementation of neutralized zeolites for the separation or purification of hydrocarbon feedstocks.

### DETAILED DESCRIPTION OF THE INVENTION

The invention relates to a process for neutralization of a cationic zeolite that is at least partially exchanged with one or more monovalent and/or multivalent cations, whereby said exchanged cationic zeolite is preferably of type X, Y, A, β, or MFI, whereby said neutralization process comprises at least the following stages:
a) Dissolution of a basic salt, preferably an alkaline salt, in an anhydrous organic solvent, preferably an anhydrous alcohol, in a very preferred manner anhydrous ethanol,
b) Degassing of the solution that is obtained at the end of stage a) by bubbling a dry inert gas,
c) Suspending the zeolite in the solution prepared in b) under dry inert gas,
d) Filtering and washing the solid that is obtained at the end of stage c) by an anhydrous organic solvent, preferably an anhydrous alcohol, and in a very preferred manner, anhydrous ethanol,
e) Calcination of the solid that is obtained at the end of stage d) in the presence of oxygen and under a dry gaseous stream.

The multivalent cation(s) is/are generally divalent or trivalent cations and are generally alkaline-earth cations or lanthanides. The monovalent cation(s) is/are generally alkaline cations.

The stages a), b), c), d) and e) of the neutralization process can generally be implemented under the operating conditions described below.

### Stage a)

Stage a) is that of dissolution of a basic salt in an anhydrous organic solvent. The concentration of the basic salt is generally greater than 0.01 mol/l, preferably between 0.01 mol/l and 5 mol/l, and the temperature is between 20 and 60° C. The stage generally takes place while being stirred at a speed of between 500 and 700 rpm.

### Stage b)

Stage b) is that of degassing the solution that is obtained at the end of stage a) by bubbling a dry inert gas, preferably dry argon, and keeping the solution under dry inert gas, preferably dry argon.

### Stage c)

Stage c) is that of suspending the zeolite in the solution prepared in b), under dry inert gas, preferably dry argon, and while being stirred at a speed that is generally between 500 and 700 rpm, whereby the temperature is generally between 20 and 40° C., and the time period of the stage is generally between 1 and 24 hours.

### Stage d)

Stage d) is that of filtering and washing the solid that is obtained at the end of stage c) by an anhydrous organic solvent, preferably an anhydrous alcohol, and in a very preferred manner anhydrous ethanol.

The volume of the anhydrous organic solvent that is used is in general at least equal to the one that is used during the ion exchange stage.

Furthermore, the solid that is obtained at the end of stage d) can be stored without running the risk of its acido-basic characteristics changing.

### Stage e)

Stage e) is that of calcination of the solid that is obtained at the end of stage d) in the presence of oxygen, at a temperature that is generally between 200 and 600° C., preferably between 300 and 550° C., for a time period that is generally between 1 and 20 hours, preferably between 10 and 15 hours, under a dry gaseous stream that is between 3 and 8 1.h⁻¹.g⁻¹, and preferably under a stream of dry compressed air.

It is generally verified by adsorption of nitrogen at 77° K. that the zeolite has preserved its pore volume.

The exchange rate obtained in the monovalent and/or multivalent cation(s) is generally verified by Inductively Coupled Plasma Atomic Emission Spectrometry (ICP-AES).

The thus prepared cationic zeolite can be used in any process for separation or purification of hydrocarbon feedstocks. Among other potential applications, it is possible to cite the separation of paraxylene from an aromatic C8 fraction, the separation of linear paraffins from a kerosene fraction, the separation of linear paraffins/branched paraffins from a gasoline fraction, the separation of paraffins/olefins, the elimination of mercaptans from natural gas, the desulfurization of the FCC gasolines, and the denitration of C4-C6 feedstocks for oligomerization.

The reduction in activity can generally be demonstrated by testing the zeolite using a model reaction that involves the isomerization of 1-dodecene. Its principle, its implementation, and its exploitation are explained in the literature (V. Santos, K. Barthelet, I. Gener., C. Canaff, P. Magnoux, Microporous and Mesoporous Materials, in press).

The execution of this test, which is a batch and liquid-phase experiment, comprises the following stages:
a) Suspending 3 g of zeolite in 75 g of 95% pure 1-dodecene, provided by Aldrich. This suspension is produced in a three-neck flask.
b) Degassing the suspension that is prepared in a) for several minutes by bubbling argon, and then holding the latter under argon inert atmosphere but without bubbling.
c) Putting under magnetic stir at 500 rpm and under heating in a silicone oil bath at 150° C. for 24 hours.
   The three-neck flask is topped with a reflux cooling system for preventing any evaporation of solution during the experiment.
d) Samples of 0.05 ml at regular time intervals and analysis of the latter by gas phase chromatography on a PONA (paraffins, olefins, naphthenes, aromatic compounds) column with a diameter of 200 μm, 0.5 μm film thickness, and 50 m length.

Starting from the chromatograms, the compositions of each sample of 1-dodecene and its different isomers (2-dodecene, 3-dodecene, 4-dodecene, 5-dodecene and 6-dodecene) are determined from which the conversion of 1-dodecene is calculated according to the following equation:

$$Conv ( 1 \text{-} dodecene ) = - \frac{{n ( 1 \text{-} dodecene )}_{initial} - {n ( 1 \text{-} dodecene )}_{final}}{{n ( 1 \text{-} dodecene )}_{initial}} \times 100.$$

It is then possible to trace the curve of the conversion into 1-dodecene based on the reaction time, the slope of the tangent to the first points of this curve corresponding to the initial speed of the isomerization reaction of 1-dodecene and reflecting the initial activity of the tested zeolite, i.e., its number of active sites relative to a reaction that involves acid sites.

### EXAMPLES

An NaCaY zeolite with an approximately 25% exchange rate is prepared by ion exchange according to the prior art starting from an NaY zeolite in powder form in aqueous medium. For this purpose, approximately 10 g of the zeolite is put directly in suspension in 1 l of 0.6 g/l of CaCl₂ solution (solution prepared starting from CaCl₂.2H₂O from Aldrich). Then, the flask is heated using a 60° C. silicone bath, and the suspension is kept under magnetic stirring. A cooling system is adapted so as to prevent the evaporation of the suspension during the exchange. The exchange lasts for approximately 7 hours.

After the exchange, the zeolite is filtered, washed with distilled water, and dried in an oven at 110° C. Then, it is dehydrated under a stream of nitrogen (3 1.h⁻¹g_{zeol}⁻¹) in a tubular furnace at 450° C. for 2 hours so as to eliminate the water that is adsorbed in the zeolite during the exchange.

This zeolite, denoted NaCaY-26%, is subjected to two basic washing cycles, one in aqueous medium and the other in anhydrous ethanol according to this invention.

For the washing by an aqueous alkaline solution, 10 g of zeolite is suspended in 250 ml of a solution that is prepared by dissolution of 4.1 g of NaOH pellets in 1 l of distilled water (0.1 mol/l concentration), and the system is placed under magnetic stifling at 500 rpm for 4 hours at ambient temperature, and then the solid is filtered and activated under a stream of nitrogen at 450° C. for 2 hours in a column. The recovered solid is denoted NaCaY-26%-NaOH 0.1 M (water).

The basic washing in alcoholic medium is implemented as described in this invention. A basic NaOH solution is prepared by dissolution of 4.1 g of NaOH pellets in 1 l of anhydrous ethanol after bubbling argon into the latter. Then, 10 g of zeolite is suspended in 250 ml of this solution. The whole mixture is put on magnetic stir at 500 rpm at 25° C. for 4 hours. After the solid is filtered and recovered, the latter is calcined at 550° C. for 2 hours under a stream of dry compressed air. The recovered solid is denoted NaCaY-26%-NaOH 0.1 M (ethanol).

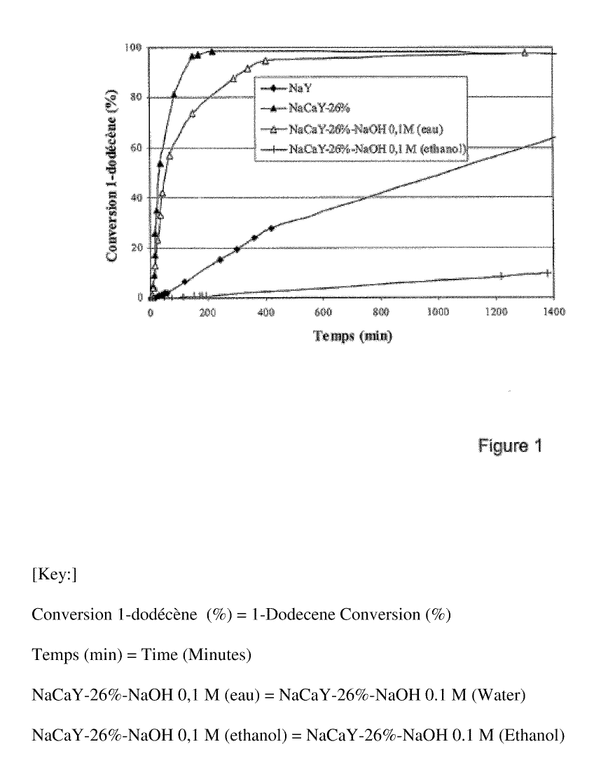

The residual acidity of these three solids, NaCaY-26%, NaCaY-26%-NaOH 0.1 M (water) and NaCaY-26%-NaOH 0.1 M (ethanol), is determined via a model transformation reaction of an olefin (1-dodecene) that makes it possible to characterize the low activities. The differences in activity of the three NaCaY between one another and the initial NaY are presented in FIG. 1, which shows the variation of the conversion of the 1-dodecene as a function of time for the NaY (initial zeolite), NaCaY-26% (solid that is not treated by a basic solution), NaCaY-26%-NaOH 0.1 M (water) and NaCaY-26%-NaOH 0.1 M (ethanol).

| | Initial | End-of-Conversion | Number of |
|---|---|---|---|
| | Speed | Test (24 hours) | Isomers |
| Solid | (mol · h⁻¹ · g_{zeol}⁻¹) | % | Formed |
| NaY | 6 · 18 · 10⁻³ | 64 | 4 |
| NaCaY-26% | 1 · 28 · 10⁻¹ | 100 | 10 |
| NaCaY-26%-NaOH | 1 · 04 · 10⁻¹ | 100 | 10 |
| 0.1M (Water) | | | |
| NaCaY-26%-NaOH | 6 · 79 · 10⁻⁵ | 10 | 4 |
| 0.1M (Ethanol) | | | |

It can be noted that the activity of the NaCaY zeolite is not reduced when it undergoes an alkaline washing in aqueous medium, but it is reduced when it is subjected to a washing of the same type in non-aqueous alcoholic medium (initial reaction speeds, conversion and number of products formed—very close). The activity becomes even lower than that of the initial NaY zeolite.

1. A process for neutralization of a cationic zeolite that is at least partially exchanged with one or more monovalent and/or multivalent cations, whereby said neutralization process comprises at least the following stages:
a) Dissolution of a basic salt, in an anhydrous organic solvent,
b) Degassing of the solution that is obtained at the end of stage a) by bubbling a dry inert gas,
c) Suspending the at least partially exchanged cationic zeolite in the solution prepared in b) under dry inert gas,
d) Filtering and washing the solid that is obtained at the end of stage c) with an anhydrous organic solvent,
e) Calcination of the solid that is obtained at the end of stage d) in the presence of oxygen and under a dry gaseous stream.

2. A process for neutralization according to claim 1, in which the anhydrous organic solvent is an anhydrous alcohol.

3. A process for neutralization according to claim 1, in which the anhydrous organic solvent is anhydrous ethanol.

4. A process for neutralization according to claim 1, said cationic zeolite being at least partially exchanged with said multivalent cation(s) which is/are alkaline-earth cations or lanthanides.

5. A process for preparation according to claim 1, said cationic zeolite being at least partially exchanged with said, monovalent cation(s) which are alkaline cations.

6. A process for neutralization according to claim 1, in which the exchanged cationic zeolite is of type X, Y, A, β, or MFI.

7. A process for neutralization according to claim 1, in which stages a), b), c), d) and e) are executed under the following operating conditions:
a) Dissolution of a basic salt, in an anhydrous organic solvent, whereby the concentration of the basic salt is between 0.01 and 1 mol/l, the temperature is between 20 and 60° C., and the stage takes place while being stirred at a speed of between 500 and 700 rpm,
b) Degassing of the solution that is obtained at the end of stage a) by bubbling a dry inert gas and keeping the solution under dry inert gas,
c) Suspending the zeolite in the solution prepared in b), under dry inert gas, while being stirred at a speed of between 500 and 700 rpm, whereby the temperature is between 20 and 40° C., and the time period of the stage is between 1 and 24 hours,
d) Filtering and washing the solid that is obtained at the end of stage c) with an anhydrous organic solvent, and
e) Calcination of the solid that is obtained at the end of stage d) in the presence of oxygen, at a temperature of between 200 and 600° C., for a time period of between 1 and 20 hours, and under a dry gaseous stream of between 3 and 8 1.h⁻¹.g⁻¹.

8. A process for the zeolite separation or the zeolitic purification of hydrocarbon feedstocks, the improvement wherein the zeolite is prepared by the process of claim 1.

9. A process according to claim 8 comprising separating paraxylene from a C8 fraction.

10. A process according to claim 8 comprising separating linear paraffins from a kerosene fraction.

11. A process according to claim 8 comprising separating linear paraffins from branched paraffins.

12. A process according to claim 8 comprising separating paraffins from olefins.

13. A process according to claim 8 comprising eliminating mercaptans from natural gas.

14. A process according to claim 8 comprising desulfurization of FCC gasolines.

15. A process according to claim 8 comprising denitration of feedstocks for oligomerization.
